(19) United States
(12) Patent Application Publication
(10) Pub. No.: US 2018/0249903 A1
(43) Pub. Date: Sep. 6, 2018

(54) ENDOSCOPE LUMEN INTERNAL CLEANER AND METHODS OF USE

(71) Applicant: Clean Endo, LLC, Collierville, TN (US)

(72) Inventors: Axel STROMBERGSSON, Franklin, TN (US); Byron SMITH, Nashville, TN (US); Ryan RAMKHELAWAN, Collierville, TN (US)

(21) Appl. No.: 15/913,831

(22) Filed: Mar. 6, 2018

Related U.S. Application Data
(60) Provisional application No. 62/467,386, filed on Mar. 6, 2017.

(51) Int. Cl. A61B 1/12

(52) U.S. Cl. CPC A61B 1/122; A61B 2090/701

(57) ABSTRACT

An endoscope lumen internal cleaner and methods of use includes a flexible tube having a first tube end an a second tube end, wherein the tube is configured to travel therein the channel or lumens of a surgical instrument, one or more apertures positioned therethrough said tube and proximate said second end, a container for housing the pressurized fluid or a pump and reservoir, the container having a discharge port, wherein the discharge port in fluid communication with a valve, the first tube end is in fluid communication with the discharge port to enable the pressurized fluid to travel therethrough the flexible tube to the one or more apertures when opening the valve, a frictional cleaning device affixed to said second end.

### CROSS REFERENCE TO RELATED APPLICATIONS

To the full extent permitted by law, the present United States Non-provisional Patent Application hereby claims priority to and the full benefit of, United States Provisional Application entitled “Pressure Fluid Channel/Lumen Cleaner with Frictional Cleaning Device Head and Fluid Jets,” having assigned Ser. No. 62/467,386, filed on Mar. 6, 2017, which is incorporated herein by reference in its entirety.

### TECHNICAL FIELD

The disclosure relates generally to medical cleaning devices and more specifically it relates to internal medical cleaning devices for channels and lumens of endoscopes.

### BACKGROUND

An endoscope is a medical device used to look inside a body cavity or organ. The scope is inserted through a natural opening, such as the mouth or the rectum to perform a medical procedure called an endoscopy. Endoscopes may include a flexible insertion tube with one or more channels or lumens therein having a distal end and orifice or opening, housing a light delivery system to illuminate the organ or object under inspection, a camera to transmit images to a screen for image review and capture, and additional channel(s) to allow entry of medical instruments or manipulators, delivery of air or water, suction, biopsy retrieval and the like. Between uses from one patient to the next the endoscope, especially its internal channels and lumens must be cleaned of body fluids and particulate, and sterilized.

There is concern about the use of unclean internal channels and lumens and there role in the transmission of diseases that commonly arise in healthcare facilities, and by viruses carried in tissues and blood of prior patients, such as hepatitis B and HIV, which may be transmitted to other patients. This concern stems from the difficulty in cleaning surgical lumens by a method meticulous enough to scrub a soiled lumen, while at the same time being simple and straightforward enough to be utilized by personnel requiring a minimum amount of training, and using an apparatus which is both inexpensive and reliable. Moreover, disinfection is difficult due to the miniature design of the endoscope, hidden and/or retractable instruments, small orifices, and long narrow channels, lumens, or passageways therethrough. To prevent body fluids and particulate from drying in these remote locations some pre-sterilization cleaning approaches have been utilized.

One previous lumen cleaning approach includes merely pre-soaking used surgical instruments having channels or lumens in a detergent bath of an enzyme solution and disinfectant and scrubbing exterior with a small scrub brush prior to sterilization. One disadvantage of this approach is that small scrub brushes and mere soaking cannot thoroughly clean the internal surfaces of the channels and lumens resulting in contaminants remaining therein the surgical instruments.

Another previous lumen cleaning approach includes a lumen brush having a plurality of bristles or que-tips affixed to ends of solid cables, wire, or braided wire and the brush is pushed through a surgical instrument channel or lumen to clean and disinfect the interior. One disadvantage of this approach is that pushing a dry scrub brush through a lumen is also problematic because it may damage the interior lumen wall. Moreover, the endoscopes hidden and/or retractable instruments, small orifices, and long narrow channels, lumens, or passageways may have dried body fluids and particulate and even grossly soiled areas. These areas are hard to clean with a dry brush alone.

Therefore, it is readily apparent that there is a recognizable unmet need for an endoscope lumen internal cleaner and methods of use thereof that functions to simultaneously mechanically scrub and in combination pressure wash or jet stream with enzymatic or other cleaning fluid directly therein the internal channels or lumens of the endoscope at the point of dried body fluids and particulate to remove tough to clean dried body fluids and particulate contained inside the internal channels or lumens of the endoscope.

### BRIEF SUMMARY

Briefly described, in example embodiment, the present apparatus overcomes the above-mentioned disadvantage, and meets the recognized need for an endoscope lumen internal cleaner and methods of use, by providing a flexible tube having a first tube end an a second tube end, wherein the tube is configured to travel therein the channel or lumens of a surgical instrument, one or more apertures positioned therethrough said tube and proximate said second end, a container for housing the pressurized fluid or a pump and reservoir, the container having a discharge port, wherein the discharge port in fluid communication with a valve, the first tube end is in fluid communication with the discharge port to enable the pressurized fluid to travel therethrough the flexible tube to the one or more apertures when opening the valve, a frictional cleaning device affixed to said second end and, thus, functions to simultaneously mechanically scrub and in combination pressure wash or jet stream with enzymatic or other cleaning fluid directly therein the internal channels or lumens of the endoscope at the point of dried body fluids and particulate to remove tough to clean dried body fluids and particulate contained inside the internal channels or lumens of the endoscope.

According to its major aspects and broadly stated, the disclosure includes a flexible tube having a first tube end an a second tube end, wherein the tube is configured to travel therein the channel or lumens of a surgical instrument, one or more apertures positioned therethrough said tube and proximate said second end, a container for housing the pressurized fluid or a pump and reservoir, the container having a discharge port, wherein the discharge port in fluid communication with a valve, the first tube end is in fluid communication with the discharge port to enable the pressurized fluid to travel therethrough the flexible tube to the one or more apertures when opening the valve, a frictional cleaning device affixed to said second end.

This disclosure is designed to provide pressurized fluid or jet stream cleaning, and in some cases, simultaneous frictional cleaning device of endoscopes and other reusable medical devices containing channels or lumens or outer contaminated surfaces that has to be cleaned prior to reuse. The fluid jets are projected out of the brush/spray nozzle in several directions, including laterally, before, within, and/or after the brush, which provides direct pressure cleaning of the sidewalls of channel or lumen and other internal surfaces. Additionally, multiple brush/spray nozzle attachments can be used for pressure cleaning of hard to reach areas and for specific dried body fluids and particulate debris located internally or externally of endoscopes and other medical devices.

Single and multiple use disposable or re-usable attachment device or accessories used on endoscopes, complex surgical devices and other reusable medical devices for the purpose of providing pressure jet streams, sometimes in combination with mechanical cleaning tools such as brushes, for cleaning and scrubbing of internal channels or lumens. The device can also be used for pressurized fluid spray cleaning of internal and external surfaces that are difficult to reach with conventional cleaning instruments. The product utilizes various accessories to achieve its cleaning action on specific medical devices. Examples include the internal jet stream which combines mechanical brushing with fluid jets, and also the multi jet stream clip-on nozzle for cleaning of duodenoscope elevator mechanisms.

This disclosure is designed to be used in various reprocessing steps, such as the pre-cleaning, and cleaning procedures of medical devices containing lumens, channels, passageways or other hard to clean surfaces whether internal or external, and other surfaces requiring different types of cleaning. In relation to the pre-cleaning step, the disclosure will also allow for point of use cleaning, basically directly after a procedure without the need of transporting the device to commence the pre-cleaning step, including internal jet stream and mechanical brushing, and thus provide a more efficient device, system, and method for cleaning internal passageways and surfaces of endoscopes and other reuseable medical devices. Additionally, alternative nozzles allow for cleaning of exterior surfaces of contaminated medical devices with or without lumens. The disclosure utilizes fluid and compressed gas propellant compressed within aerosol can, micro catheters with one or more bristle or silicone or other material brush(es) attached in conjunction with one or more spray nozzles whether positioned in front of, within, or behind, a rotary brush connected to speed cable and powered by battery driven micro motor. Variants of the theme includes rigid catheters with multi brush sizes for use on various sized medical devices, as well as other accessories such as a clip-on nozzle head for specific cleaning of the elevator mechanism of duodenoscopes. The disclosure can also be powered by AC, DC plugin or battery powered fluid pumps that provide continuous, intermittent, pulsatile, variable and adjustable fluid flow. Also, the disclosure can use UV light positioned proximate a second end of the tube, whether internally and/or externally for additional disinfection benefits.

In a preferred embodiment, a system to clean one or more channels therein a surgical instrument, the system includes a tube having a first tube end an a second tube end, said tube configured to travel therein the channel of the surgical instrument, one or more discharge apertures positioned therethrough said tube and proximate said second tube end, and a container for housing a pressurized medium, said container having a discharge port, said discharge port in communication with a valve, and said first tube end in communication with said discharge port, to enable said pressurized medium to travel therethrough said tube to said one or more discharge apertures when opening said valve.

In still a further exemplary embodiment, a method of cleaning one or more channels therein a surgical instrument, the method includes providing a tube having a first tube end an a second tube end, said tube configured to travel therein the channel of the surgical instrument, one or more discharge apertures positioned therethrough said tube and proximate said second tube end, and a container for housing a pressurized medium, said container having a discharge port, said discharge port in communication with a valve, and said first tube end in communication with said discharge port, and discharging said pressurized medium to enable said pressurized medium to travel therethrough said tube to said one or more discharge apertures to clean the one or more channels therein the surgical instrument.

Accordingly, a feature of the endoscope lumen internal cleaner and methods of use is its ability to functions to simultaneously mechanically scrub and in combination pressure wash or jet stream with enzymatic or other cleaning fluid directly therein the internal channels or lumens of the endoscope at the point of dried body fluids and particulate to remove tough to clean dried body fluids and particulate contained inside the internal channels or lumens of the endoscope.

Another feature of the endoscope lumen internal cleaner and methods of use is its ability to reduce or prevent damage to the internal channels or lumens of the endoscope by providing a liquid medium directly to the area being mechanically scrubbed. Cost reduction by preventing internal channels or lumens of the endoscope damage.

Still another feature of the endoscope lumen internal cleaner and methods of use is its ability to reduce the amount of cleaning or soaking fluid, such as an enzymatic fluid needed since the cleaning fluid is provided directly to the area being mechanically scrubbed rather than the amount of fluid necessary to soak the entire endoscope.

Yet another feature of the endoscope lumen internal cleaner and methods of use is its ability to provide pressurized fluid cleaning of channels/lumens and external surfaces of endoscopes and other medical devices or mechanical brushing.

Yet another feature of the endoscope lumen internal cleaner and methods of use is its ability to provide faster channel/lumen/surface cleaning after the medical procedure, i.e. at bedside—immediate pre-cleaning at point of use, before body fluids and particulate contained inside the internal channels or lumens of the endoscope may dry.

Yet another feature of the endoscope lumen internal cleaner and methods of use is its ability to provide assisted cleaning provided by micro bubbles in high-pressure fluid or enzymatic fluids directly therein the internal channels or lumens of the endoscope at the point of dried body fluids and particulate.

Yet another feature of the endoscope lumen internal cleaner and methods of use is its ability to provide sonic fluid agitation to assist with removal of bioburden accumulated directly therein the internal channels or lumens of the endoscope at the point of dried body fluids and particulate.

Yet another feature of the endoscope lumen internal cleaner and methods of use is its ability to provide battery powered rotary motion to the flexible tube and ultimately rotary brush head allows for enhanced scrubbing of internal channels or lumens of the endoscope at the point of dried body fluids and particulate.

Yet another feature of the endoscope lumen internal cleaner and methods of use is its ability to provide a single trigger design to activate pressurized fluid cleaning and/or rotary brush head allowing for ease of use and operation.

Yet another feature of the endoscope lumen internal cleaner and methods of use is its ability to provide a single use disposable fluid tube and brush head design to reduce the potential of cross contamination between surgical instruments.

Yet another feature of the endoscope lumen internal cleaner and methods of use is its ability to provide pressurized fluid and micro bubbles directly therein the internal channels or lumens of the endoscope at the point of dried body fluids and particulate to remove debris trapped in cracks and groves and from internal surfaces of the internal channels or lumens of the endoscope.

Yet another feature of the endoscope lumen internal cleaner and methods of use is its ability to provide a pressurized fluid source of cleaning fluid, such as an enzymatic fluid spray to help remove debris and other contamination from exterior surfaces of the endoscope or other medical instrument.

Yet another feature of the endoscope lumen internal cleaner and methods of use is its ability to provide interchangeable accessories that attach to the head or valve of pressurized fluid source, catheter, or fluid tube, allowing for various additional cleaning tools to be connected and disconnected as needed during the reprocessing cycle.

Yet another feature of the endoscope lumen internal cleaner and methods of use is its ability to reduce the likelihood of dried bioburden and biofilm formation therein the endoscope.

Yet another feature of the endoscope lumen internal cleaner and methods of use is its ability to improve cleaning of channels/lumens and external surfaces of endoscopes and other medical devices via mechanical frictional cleaning device and/or pressurized fluid.

These and other features of the endoscope lumen internal cleaner and methods of use will become more apparent to one skilled in the art from the following Detailed Description of the Embodiments and Claims when read in light of the accompanying drawing Figures.

### BRIEF DESCRIPTION OF THE DRAWINGS

The present endoscope lumen internal cleaner and methods of use will be better understood by reading the Detailed Description of the embodiments with reference to the accompanying drawing figures, in which like reference numerals denote similar structure and refer to like elements throughout, and in which:

FIG. 1 is a top view of an exemplary embodiment of an endoscope lumen internal cleaning system;

FIG. 2 is a top perspective view of an exemplary embodiment of a friction cleaning device of the endoscope lumen internal cleaning system of FIG. 1;

FIG. 3 is a side perspective view of an exemplary internal lumen cleaning apparatus accessory;

FIG. 4A is a side view of an alternate exemplary internal lumen cleaning apparatus accessory;

FIG. 4B is a side view of an alternate exemplary internal lumen cleaning apparatus accessory;

FIG. 4C is a side view of an alternate exemplary internal lumen cleaning apparatus accessory;

FIG. 4D is a side view of an alternate exemplary internal lumen cleaning apparatus accessory;

FIG. 5 is a front perspective view of an exemplary endoscope having a pair of channels or lumens, shown with channels or lumens being cleaned;

FIG. 6A is a top cross-sectional view of an alternate exemplary embodiment of an endoscope lumen internal cleaning system, including a rotary pack;

FIG. 6B is a top cross-sectional view of an alternate exemplary embodiment of an endoscope lumen internal cleaning system, including a rotary pack; and

FIG. 7 is a flow diagram of a method sport racket score keeping and vibration dampening.

It is to be noted that the drawings presented are intended solely for the purpose of illustration and that they are, therefore, neither desired nor intended to limit the disclosure to any or all of the exact details of construction shown, except insofar as they may be deemed essential to the claimed invention.

### DETAILED DESCRIPTION

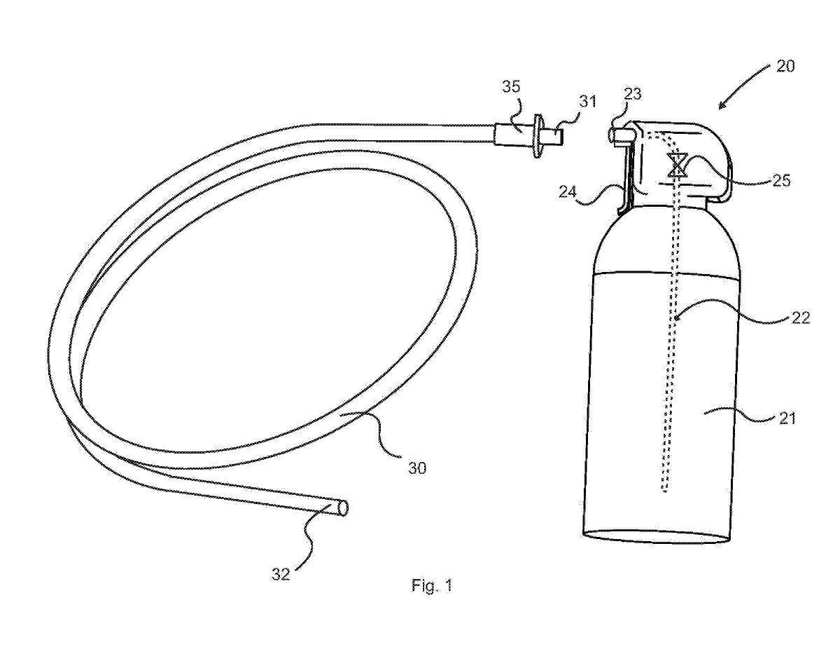

In describing the exemplary embodiments of the present disclosure, as illustrated in FIGS. 1-3, 4A-4D, 5, 6A, 6B, 7, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples.

Referring now to FIG. 1, by way of example, and not limitation, there is illustrated an example embodiment endoscope lumen cleaning system 10. Endoscope lumen cleaning system 10 may include a pressurized fluid or air source, such pressurized medium 20 from a pump and reservoir or container to pressurize pressurized medium 20, a pressurized container, such as aerosol canister 21. Aerosol canister 21 may include a reservoir or vessel capable of holding a fluid under pressure. At one end of aerosol canister 21 may be a cap, such as release mechanism 22 to discharge pressurized fluids therefrom aerosol canister 21. Release mechanism 22 may further include valve 25, finger trigger 24 attached thereto valve 25, and discharge port 23.

Endoscope lumen cleaning system 10 may further include a plastic catheter or other conduit, rigid, semi-rigid, or flexible, such as tube 30 capable of traversing a narrow passageways and transport pressurized fluid or jet stream therein. Flexible tube 30 may include first tube end 31 and second tube end 32, wherein first tube end 31 is preferably attached, friction fit, or removably affixed thereto discharge port 23 of pressurized medium 20. It is contemplated herein that first tube end 31 may include an adapter-connector therebetween first tube end 31 and discharge port 23 of pressurized medium 20, such as discharge connector 35. Second tube end 32 may include internal fluid or air lumen cleaning accessory 40 having a hole, spray nozzle, fluid jets, or other pressure release point(s), such as discharge aperture 50 proximate second tube end 32 to provide one or more directional delivery ports of pressurized fluid. It is contemplated herein that discharge aperture 50 may include pressure release point, hole or aperture therein second tube end 32, attachable accessories thereto second tube end 32, such as spray nozzle, fluid jets or other pressure release mechanisms.

It is contemplated herein that pressurized fluid may discharge therefrom discharge aperture 50 at an angle A being acute, perpendicular, obtuse, and parallel thereto second tube end 32.

It is further contemplated herein that discharge port 23 may include a friction fitting, snap fitting, or other coupling means known by one of ordinary skill in the art.

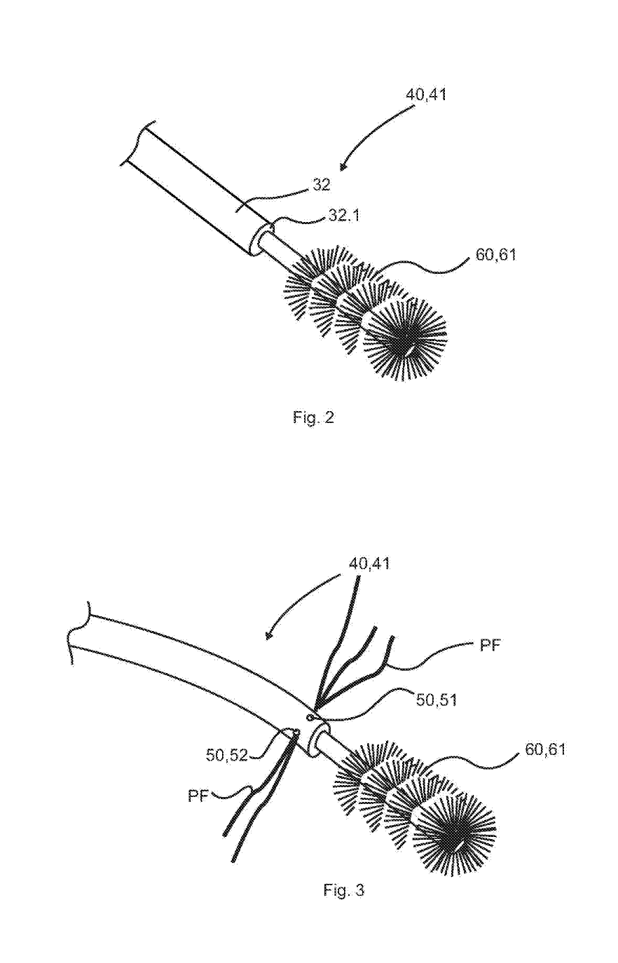

Referring now to FIGS. 2 and 3, by way of example, and not limitation, there is illustrated an example embodiment lumen cleaning accessory 40. Lumen cleaning accessory 40 may include brush head, wipe, swipe, plunger, lilipad plunger, corkscrew plunger, bristle brush, spiral brush, or other friction cleaning device 60, and more specifically brush head 61. In this embodiment, brush head 61 is preferably affixed thereto tube end 32.1 of second tube end 32 and discharge aperture 50 is preferably positioned behind brush head 61 and proximate second tube end 32. As shown in FIG. 3, in use trigger 24 is actuated to open valve 25, discharging effervescent, enzymatic or other cleaning agent, sonic, pulsating, such as pressurized fluid (or air) PF out discharge port 23 and into first tube end 31. Pressurized fluid PF traverse therein tube 30 thereto second tube end 32 and discharges therefrom second tube end 32 of tube 30 via discharge aperture 50, such as first discharge aperture 51, second discharge aperture 52, and third discharge aperture 53 positioned there behind friction cleaning device 60.

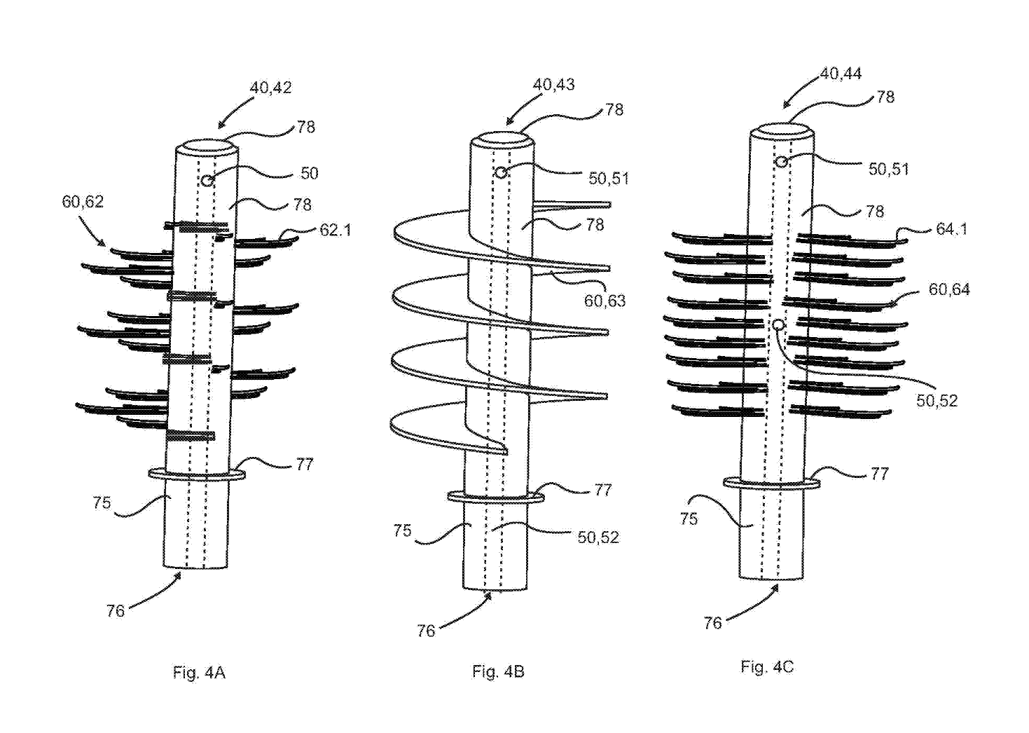

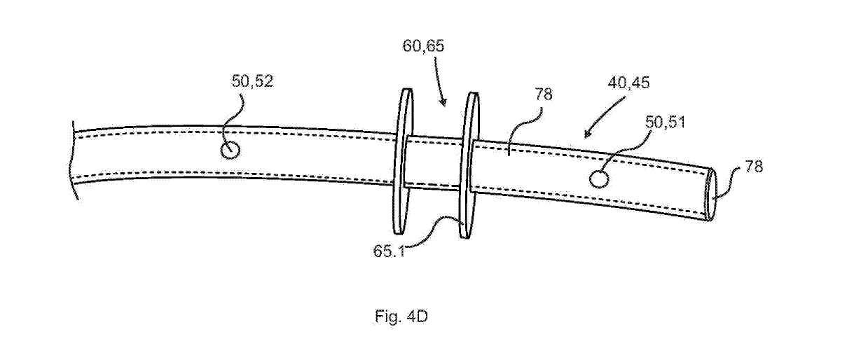

Referring now to FIGS. 4A, 4B, 4C and 4D, by way of example, and not limitation, there is illustrated a set of alternate embodiment one or more mechanical lumen cleaning accessory 40. One or more mechanical lumen cleaning accessory 40 may include a variety of discharge aperture(s) 50 and/or in combination a variety of friction cleaning device(s) 60. Referring again to FIGS. 4A, 4B, 4C and 4D, lumen cleaning accessory 40 may include accessory connector 75 having accessory input port 76 configured to connect, attach, or removeably affix one or more mechanical lumen cleaning accessory 40 thereto second tube end 32 of tube 30. Moreover, accessory connector 75 may include a gripping mechanism, such as flared disc 77 positioned thereon accessory tube 78 and configured to enable finger gripping of accessory connector 75 to assist in removal or attachment of one or more mechanical lumen cleaning accessory 40 thereto second tube end 32 of tube 30. Furthermore, one or more mechanical lumen cleaning accessory 40 may be configured as a hollow tube or conduit to enable pressurized fluid PF to traverse therethrough one or more mechanical lumen cleaning accessory 40 to discharge aperture 50, and one or more mechanical lumen cleaning accessory 40 may include a cap end 78 to close off lumen cleaning accessory 40.

Referring again to FIG. 4A, mechanical lumen cleaning accessory 42 may include friction cleaning device 60, and more specifically friction cleaning head 62 having individual or sets of different length bristles 62.1 extending therefrom accessory tube 78 and configured in a spiral or lily pad formation therearound accessory tube 78. In this embodiment, discharge aperture 50 may be positioned in front of mechanical lumen cleaning accessory 42 and proximate cap end 78 or in front of or on the leading edge of friction cleaning device 62.

Referring again to FIG. 4B, lumen cleaning apparatus accessory 43 may include friction cleaning device 60, and more specifically friction cleaning head 63 having individual or sets of spiral paddles 63.1 extending therefrom accessory tube 78 and configured in a spiral or cork screw formation therearound accessory tube 78. In this embodiment, first discharge aperture 51 may be positioned proximate cap end 78 or in front of or on the leading edge of friction cleaning device 63 and second discharge aperture 52 may be positioned proximate accessory connector 75 or flared disc 77 or behind of, or back of, or on the trailing edge of friction cleaning device 63.

Referring again to FIG. 4C, mechanical lumen cleaning accessory 44 may include friction cleaning device 60, and more specifically friction cleaning head 64 having individual or sets of bristles 64.1 extending therefrom accessory tube 78 and configured in a bundle or compact bristle formation therearound accessory tube 78. In this embodiment, first discharge aperture 51 may be positioned proximate cap end 78 in front of or on the leading edge of friction cleaning device 64 and second discharge aperture 52 may be positioned therein individual or sets of bristles 64.1 of friction cleaning device 64 or formed integral therein friction cleaning device 64.

Referring again to FIG. 4D, mechanical lumen cleaning accessory 45 may include friction cleaning device 60, and more specifically friction cleaning head 65 having individual or sets of disc 65.1 extending therefrom accessory tube 78 and configured in a one or more series of disc formation therearound accessory tube 78. In this embodiment, first discharge aperture 51 may be positioned proximate cap end 78 in front of or on the leading edge of friction cleaning device 65 and second discharge aperture 52 may be positioned behind of, in back of, or on the trailing edge of friction cleaning device 65.

It is contemplated herein that a variety of configurations and layouts of friction cleaning device 60, discharge aperture 50, and combinations thereof may be utilized depending on the surgical instrument channel or lumen to be cleaned and disinfected and the body fluids and particulate to be cleaned.

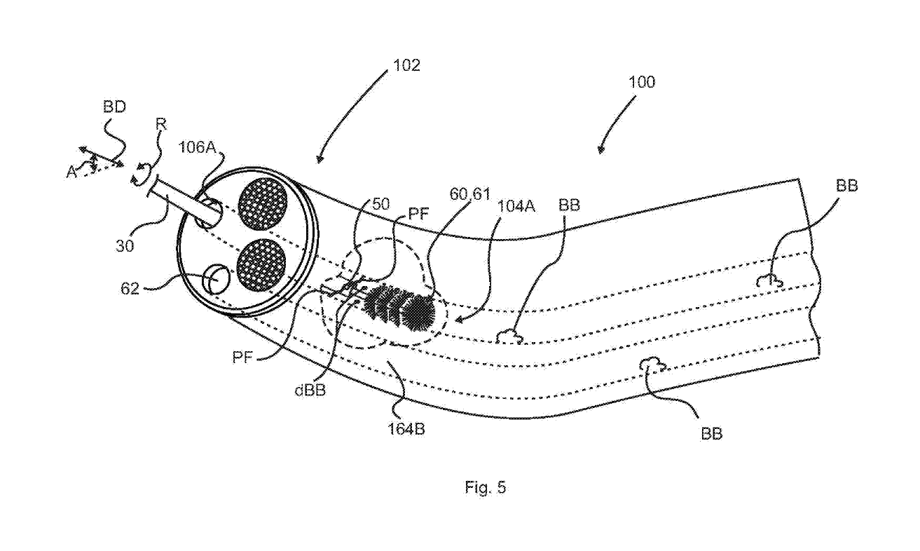

Referring now to FIG. 5, by way of example, and not limitation, there is illustrated an example embodiment of distal tip 102 of an endoscope 100. Distal tip 102 of an endoscope 100 may include one or more internal channels, lumens, or passageways, such as endoscope conduit 104 traversing internally the length of flexible endoscope 100. One or more internal conduits 104 such as first conduit 104A having an first endoscope aperture 106A and second conduit 104B having an second endoscope aperture 106B, which may include conduits and apertures for delivery of water, pressurized water, air, compressed air, delivery of vacuum, optic image bundle, image sensor or capture device, fiber optic bundle, light source, delivery of medical instrument(s), such as a suture to conduct a biopsy or other medical instrument to meet the needs of the medical application being performed with endoscope 100.

It is known by one of ordinary skill in the art that disinfection of endoscope 100 is difficult due to the miniature design of the endoscope 100, hidden and/or retractable instruments, one or more endoscope aperture 106, and one or more long narrow channels, lumens or passageways, such as endoscope conduit 104. To prevent body fluids, dried body fluids, tissue, cells, and particulate (bioburden BB) from drying in these remote locations after use of endoscope 100 a pre-cleaning apparatus, such as endoscope lumen cleaning system 10 may be utilized.

In use, second tube end 32 of tube 30 of endoscope lumen cleaning system 10 may be inserted therein endoscope conduit 104 and friction cleaning device 60 attached thereto or configured therein tube 30 may be slid, twisted, or pushed therein as well as slid in-and-out, direction of travel, or in a back-and-forth direction BD to friction clean and scrub bioburden BB therefrom endoscope conduit 104. Moreover, alone or in combination therewith friction cleaning device 60, second tube end 32 of tube 30 of endoscope lumen cleaning system 10 may be inserted therein endoscope conduit 104 and discharge aperture 50 attached thereto or configured therein tube 30 may be slid or pushed therein as well as slid in-and-out or in a back-and-forth direction BD to discharge pressurized fluid PF from tube 30 to pressure wash or jet stream clean bioburden BB therefrom endoscope conduit 104.

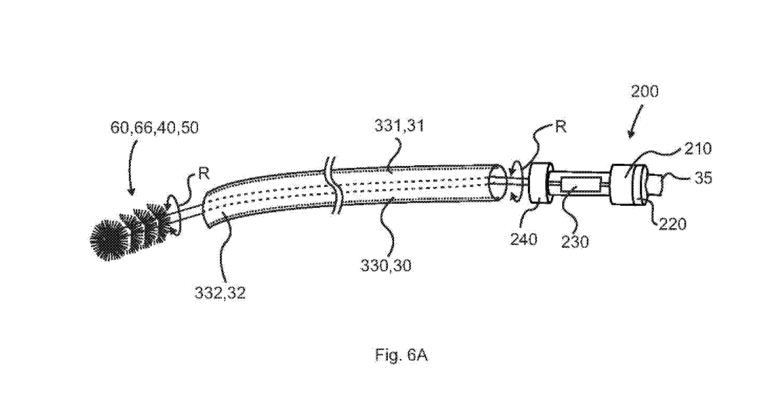

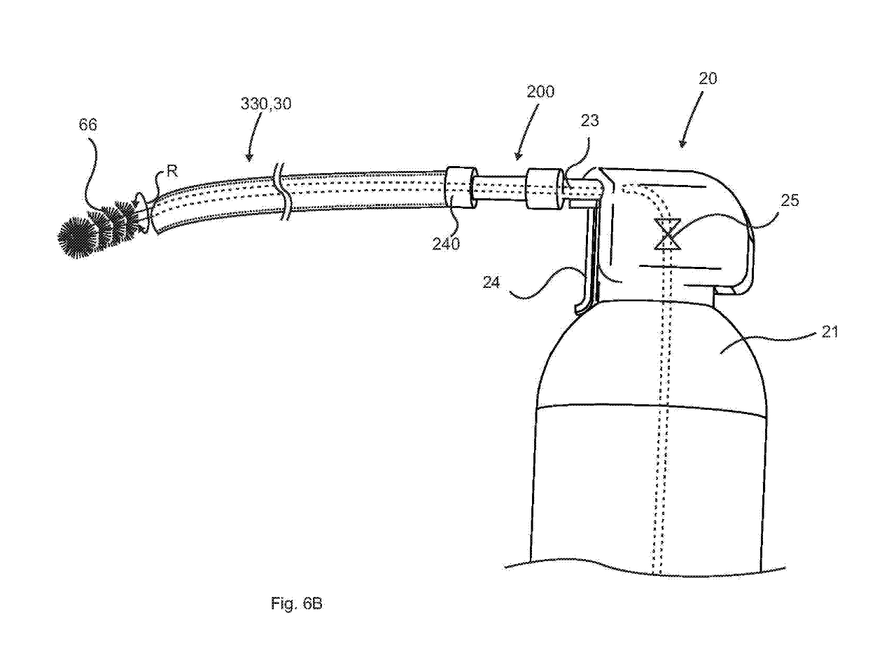

Referring now to FIG. 6A and 6B, by way of example, and not limitation, there is illustrated an alternate exemplary embodiment of a rotational device, such as rotary pack 300. Rotary pack 300 may include a micro catheter, wire, braided wire or the like, whether plastic, rigid, semi-rigid, or flexible, such as micro cable 330 capable of traversing a narrow passageways, applying a rotational force, and/or transport pressurized fluid or jet stream therein. Micro cable 330 may include first micro cable end 331 and second micro cable end 332, wherein first micro cable end 331 is preferably attached, friction fit, or removably affixed thereto discharge port 23 of pressurized medium 20. It is contemplated herein that first micro cable end 331 may include an adapter-connector therebetween first micro cable end 331 and discharge port 23 of pressurized medium 20, such as discharge connector 35.

Moreover, first micro cable end 331 may include a rotational device, electric motor, hydraulic turbine, or the like, such as rotary pack 200 capable of rotating micro cable 330 and friction cleaning device 60, 66. Rotary pack 200 may include micro motor 210 (to provide rotational force to the rotary pack 200), electrically connected to power source 220, and coupler 230 configured to connect motor 210 to first micro cable end 331. Micro motor 210, power source 220, and coupler 230 may be in cased or housed in a protective housing 240. It is contemplated herein that micro cable 330 may be a hollow tube or conduit to direct or carry pressurized fluid PF from first micro cable end 331 and second micro cable end 332. Alternatively, micro cable 330 may pass therethrough internal passageways of flexible tube 30 with pressurized fluid PF from first tube end 31 to second tube end 32 and connection thereto rotary brush head 66.

Second micro cable end 332 may include internal lumen cleaning accessory 40 having a hole, spray nozzle, fluid jets, or other pressure release point(s), such as discharge aperture 50 proximate second micro cable end 332 to provide one or more directional delivery ports of pressurized fluid. Moreover, one or more mechanical lumen cleaning accessory 40 may include brush head, wipe, swipe, plunger, lilipad plunger, corkscrew plunger, bristle brush, spiral brush or other friction cleaning device 60, and more specifically rotary brush head 66. In this embodiment, rotary brush head 66 is preferably affixed thereto second micro cable end 332 and discharge aperture 50 is preferably positioned behind, within, or in front of rotary brush head 66 and proximate second micro cable end 332.

In use, and as shown in FIG. 6B, on/off switch, such as trigger 24 is actuated to electrically connect micro motor 210 to power source 22 (or discharge pressurized fluid PF through a turbine) to rotate coupler 230 rotationally connected to first micro cable end 331 (first tube end 31) and, thus, rotating micro cable 330 (tube 30) and second micro cable end 332 (second tube end 32) connected thereto mechanical friction cleaning device 60, 66 resulting in a rotational spin R therein endoscope conduit 104, as shown in FIG. 5. Rotational spin R of rotary brush head 66 may rotary friction clean and scrub bioburden BB therefrom endoscope conduit 104.

As shown in FIG. 6B, in use trigger 24 is actuated to open valve 25, discharging pressurized fluid PF out discharge port 23, therethrough or around rotary pack 200 and into first tube end 31. Pressurized fluid PF traverse therein tube 30 thereto second tube end 32 and discharges therefrom second tube end 32 of tube 30 via discharge aperture 50, such as first discharge aperture 51, second discharge aperture 52, and third discharge aperture 53 positioned there behind friction cleaning device 60 rotating micro cable 330 and second tube end 32 connected thereto mechanical friction cleaning device 60, 66 resulting in a rotational spin R therein endoscope conduit 104, as shown in FIG. 5. Rotational spin R of rotary brush head 66 may rotary friction clean and scrub and power wash or flush bioburden BB therefrom endoscope conduit 104.

It is contemplated herein that rotary pack 200 may be formed integral thereto tube 30 rather than formed as a separate component to be releasably affixed thereto tube 30.

It is further contemplated herein that discharge port 23 or discharge connector 35 may be sized or configured adjustable to accommodate different diameter or sized tube 30.

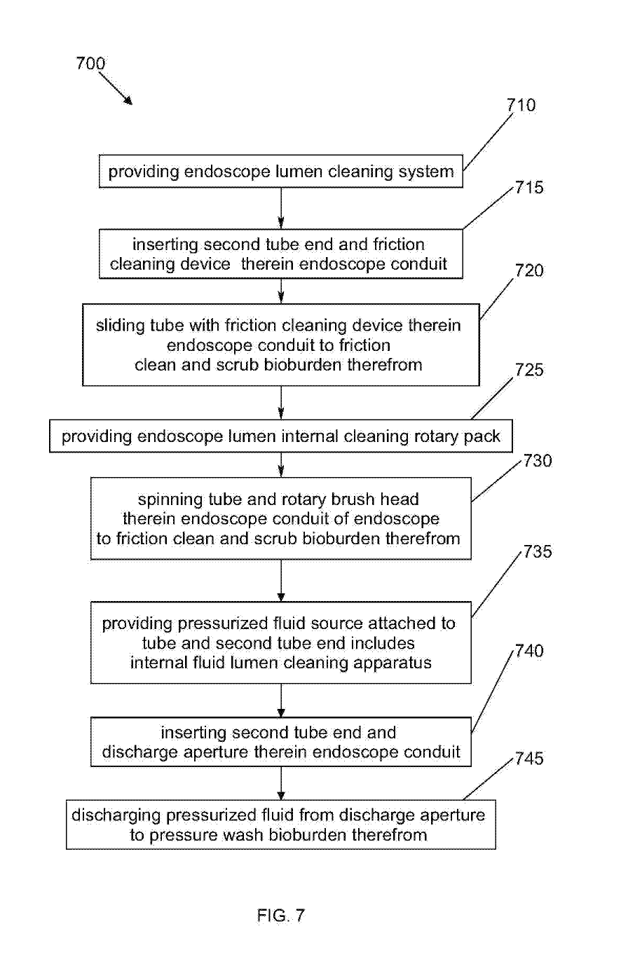

Referring now to FIG. 7, there is illustrated a flow diagram 700 of utilizing endoscope lumen cleaning system 10 to mechanically scrub and/or pressure wash or jet stream with enzymatic or other cleaning fluid directly the internal channels or lumens of the endoscope at the point of dried body fluids and particulate to remove tough to clean dried body fluids and particulate contained inside the internal channels or lumens, endoscope conduit 104 of endoscope 100. In block or step 710, providing endoscope lumen cleaning system 10 including tube 30 having first tube end 31 and second tube end 32, with friction cleaning device 60 preferably affixed thereto tube end 32 as described above in FIGS. 1-6. In block or step 715, inserting second tube end 32 of tube 30 and friction cleaning device 60 of endoscope lumen cleaning system 10 therein endoscope conduit 104 of endoscope 100. In block or step 720, sliding, twisting, or pushing tube 30 therein, in-and-out, rotating, or in a back-and-forth direction BD to friction clean and scrub bioburden BB via like movement of attached friction cleaning device 60 therein endoscope conduit 104 of endoscope 100.

In block or step 725, providing endoscope lumen internal cleaning rotary pack 300. In block or step 730, sliding, twisting, rotating, spinning, or pushing tube 30 therein via endoscope lumen internal cleaning rotary pack 300, in-and-out, rotating, in a back-and-forth direction BD, rotational spin R to friction clean and rotary scrub bioburden BB via like movement of attached rotary brush head 66 therein endoscope conduit 104 of endoscope 100.

In block or step 735, providing pressurized medium 20 attached to tube 30 having first tube end 31 and second tube end 32, wherein first tube end 31 is preferably attached, friction fit, or removably affixed thereto discharge port 23 of pressurized medium 20 and second tube end 32 may include internal fluid or air lumen cleaning accessory 40 having a hole, spray nozzle, fluid jets, or other pressure release point(s), such as discharge aperture 50 proximate second tube end 32, as described above in FIGS. 1-6. In block or step 740, inserting second tube end 32 of tube 30 and discharge aperture 50 of endoscope lumen cleaning system 10 therein endoscope conduit 104 of endoscope 100. In block or step 745, discharging pressurized fluid PF from discharge aperture 50 proximate second tube end 32 of tube 30 to pressure wash, flush, or jet stream clean bioburden BB therefrom endoscope conduit 104 of endoscope 100.

Moreover, endoscope lumen cleaning system 10 may be designed to be used in various reprocessing steps, such as the pre-cleaning, and cleaning procedures of medical devices containing lumens, channels, passageways or other hard to clean surfaces whether internal or external, and other surfaces requiring different types of cleaning. In relation to the pre-cleaning step, the disclosure will also allow for point of use cleaning, basically directly after a procedure without the need of transporting the device to commence the pre-cleaning step, including internal jet stream and mechanical brushing, and thus provide a more efficient device, system, and method for cleaning internal passageways and surfaces of endoscopes and other reuseable medical devices. Additionally, alternative nozzles allow for cleaning of exterior surfaces of contaminated medical devices with or without lumens. The disclosure utilizes pressurized fluid PF and/or compressed gas propellant compressed within aerosol canister 21, micro catheters with one or more bristle or silicone or other material brush(es), of friction cleaning device(s) 60 attached in conjunction with one or more spray nozzles, discharge aperture 50 whether positioned in front of, within, or behind, a rotary brush, rotary brush head 66 connected to speed cable and powered by battery driven micro motor, rotary pack 300.

Variants of the theme includes rigid catheters with multi brush sizes for use on various sized medical devices, as well as other accessories such as a clip-on nozzle head for specific cleaning of the elevator mechanism of duodenoscopes. The disclosure can also be powered by AC, DC plugin or battery powered, rotary pack 300 fluid pumps that provide continuous, intermittent, pulsatile, variable and adjustable fluid flow. Also, the disclosure can use UV light 78 positioned proximate a second end of the tube, second tube end 32 whether internally and/or externally for additional disinfection benefits.

The foregoing description and drawings comprise illustrative embodiments of the present disclosure. Having thus described exemplary embodiments, it should be noted by those ordinarily skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments of the disclosure will come to mind to one ordinarily skilled in the art to which this disclosure pertains having the benefit of the teachings presented in the foregoing descriptions and the associated drawings. Although specific terms may be employed herein, they are used in a generic and descriptive sense only and not for purposes of limitation. Moreover, the present disclosure has been described in detail, it should be understood that various changes, substitutions and alterations can be made thereto without departing from the spirit and scope of the disclosure as defined by the appended claims. Accordingly, the present disclosure is not limited to the specific embodiments illustrated herein, but is limited only by the following claims.

What is claimed is:

1. A system to clean one or more channels therein a surgical instrument, said system comprising:
a tube having a first tube end an a second tube end, said tube configured to travel therein the channel of the surgical instrument;
one or more discharge apertures positioned therethrough said tube and proximate said second tube end; and
a container for housing a pressurized medium, said container having a discharge port, said discharge port in communication with a valve, and said first tube end in communication with said discharge port, to enable said pressurized medium to travel therethrough said tube to said one or more discharge apertures when opening said valve.

2. The system of claim 1, wherein said pressurized medium is a fluid.

3. The system of claim 2, wherein said container further comprises a pump to pressurize said fluid.

4. The system of claim 1, wherein said valve further comprises a finger trigger attached thereto said valve.

5. The system of claim 1, wherein said first tube end further comprises a discharge connector configured to attach thereto said discharge port.

6. The system of claim 1, wherein said one or more discharge apertures discharges said pressurized medium at an angle selected from the group consisting of an acute, an obtuse, a perpendicular, and a parallel to said second tube end.

7. The system of claim 1, further comprises a friction cleaning device affixed to said second tube end.

8. The system of claim 7, wherein said friction cleaning device is selected from the group consisting of a brush, a wipe, a swipe, a plunger, a lilipad plunger, a corkscrew plunger, a bristle brush, a spiral brush and combinations thereof.

9. The system of claim 8, wherein said one or more discharge apertures are positioned proximate said friction cleaning device and selected from the position consisting of a front, a back, integral therein, and combinations thereof.

10. The system of claim 7, wherein said friction cleaning device and said tube are disposable.

11. The system of claim 9, further comprises a rotary pack configured to rotate said first tube end, said second tube end, and said friction cleaning device affixed to said second tube end.

12. The system of claim 11, wherein said rotary pack further comprises an electric motor.

13. The system of claim 12, wherein said rotary pack further comprises a turbine configured therein said discharge port.

14. The system of claim 11, wherein said rotary pack configured to rotate a micro cable positioned therein said tube to said second tube end, and rotate said friction cleaning device affixed to said second tube end.

15. The system of claim 1, wherein said tube is flexible.

16. A method of to clean one or more channels therein a surgical instrument, said method comprising the steps of:
providing a tube having a first tube end an a second tube end, said tube configured to travel therein the channel of the surgical instrument, one or more discharge apertures positioned therethrough said tube and proximate said second tube end, and a container for housing a pressurized medium, said container having a discharge port, said discharge port in communication with a valve, and said first tube end in communication with said discharge port; and
discharging said pressurized medium to enable said pressurized medium to travel therethrough said tube to said one or more discharge apertures to clean the one or more channels therein the surgical instrument.

17. The method of claim 16, further comprises the step of providing a friction cleaning device affixed to said second tube end.

18. The method of claim 17, further comprises the step of inserting said second tube end and said friction cleaning device therein one of the one or more channels therein the surgical instrument.

19. The method of claim 18, further comprises the step of sliding said tube in a back-and-forth direction to friction clean a bioburden therein one of the one or more channels therein the surgical instrument.

20. The method of claim 18, further comprises the step of providing a rotary pack to rotate said friction cleaning device therein one of the one or more channels therein the surgical instrument to friction clean a bioburden therein one of the one or more channels therein the surgical instrument.

21. The method of claim 18, further comprises the step of discharging a pressurized fluid therethrough said one or more discharge apertures to flush a bioburden therefrom one of the one or more channels therein the surgical instrument.
